(19) United States
(12) Patent Application Publication
(10) Pub. No.: US 2023/0110719 A1
(43) Pub. Date: Apr. 13, 2023

(54) SYSTEMS AND METHODS FOR FEW-SHOT PROTEIN FITNESS PREDICTION WITH GENERATIVE MODELS

(71) Applicant: salesforce.com, inc., San Francisco, CA (US)

(72) Inventors: Ben Krause, Palo Alto, CA (US); Ali Madani, Oakland, CA (US)

(21) Appl. No.: 17/589,623

(22) Filed: Jan. 31, 2022

Related U.S. Application Data
(60) Provisional application No. 63/252,529, filed on Oct. 5, 2021.

(51) Int. Cl. G16B 40/30; G06F 40/40; G06N 3/12

(52) U.S. Cl. CPC G16B 40/30; G06F 40/40; G06N 3/126

(57) ABSTRACT

Embodiments are directed to finetuning a pre-trained language model using generative fitness finetuning. The generative fitness finetuning reuses a probability distribution learned during unsupervised training of the pre-trained language model to finetune and assay labeled data. The generative fitness finetuning trains the language model to classify a relative fitness of protein sequence pairs based on the corresponding probability of the protein sequences in the pairs. The generative fitness finetuning identifies protein sequences in the pairs with a higher probability as also having higher fitness. The trained and finetuned language model identifies fitness of a protein sequence.

### CROSS-REFERENCE TO RELATED APPLICATIONS

This application is a nonprovisional of and claims priority under 35 U.S.C. 119 to U.S. Provisional Application No. 63/252,529, filed Oct. 5, 2021, which is hereby expressly incorporated by reference herein in its entirety.

### TECHNICAL FIELD

The embodiments relate generally to machine learning systems and protein sequencing, and more specifically to systems and methods for few-shot protein fitness prediction with generative models.

### BACKGROUND

Proteins are complex molecules that perform a spectacular variety of functions that drive biological processes. Their versatility gives them widespread medical and environment use cases. For example, a protein is encoded by a specific raw amino acid sequence, and during synthesis, this chain of amino acids folds in ways that exhibit a local (e.g., secondary) and a global (e.g., tertiary) structure. These structural properties then directly determine a unique function of the synthesized protein, e.g., to serve as part of a vaccine to certain virus, a catalyst, etc. The field of protein engineering aims to synthesize proteins that have high “fitness” for their intended function. However, existing techniques for protein engineering is still largely limited. For example, biophysical simulations that account for the structure of a protein have not yet been shown to be able to predict a protein’s fitness to perform a particular function. Directed evolution engineers proteins via artificial evolution in a laboratory, but relies on random mutations to generate candidate proteins for testing.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a simplified diagram of a computing device that implements a protein fitness prediction framework, according to some embodiments described herein

FIG. 2 is a simplified diagram illustrating a protein fitness landscape, according to some embodiments.

FIG. 3 is a simplified block diagram illustrating a protein fitness prediction module, according to some embodiments.

FIG. 4 is a simplified diagram illustrating examples of the protein fitness prediction tasks, according to some embodiments.

FIG. 5 is a simplified diagram of a method for training a pre-trained language model using a protein fitness prediction module, according to some embodiments.

FIG. 6 is a simplified diagram of a method for finetuning the language model using generative fitness finetuning, according to some embodiments.

FIG. 7 is a simplified diagram of a method for predicting protein fitness of a protein sequence, according to some embodiments.

In the figures, elements having the same designations have the same or similar functions.

### DETAILED DESCRIPTION

As used herein, the term “network” may comprise any hardware or software-based framework that includes any artificial intelligence network or system, neural network or system and/or any training or learning models implemented thereon or therewith.

As used herein, the term “module” may comprise hardware or software-based framework that performs one or more functions. In some embodiments, the module may be implemented using one or more neural networks.

Artificial intelligence, implemented with neural networks and deep learning models, has demonstrated great promise as a technique for automatically analyzing real-world information with human-like accuracy. In general, such neural network and deep learning models receive input information and make predictions based on the same. Whereas other approaches to analyzing real-world information may involve hard-coded processes, statistical analysis, and/or the like, neural networks learn to make predictions gradually, by a process of trial and error, using a machine learning process. A given neural network model may be trained using a large number of training examples, proceeding iteratively until the neural network model begins to consistently make similar inferences from the training examples that a human might make. Neural network models have been shown to outperform and/or have the potential to outperform other computing techniques in a number of applications.

In one embodiment, artificial intelligence (Al) models may be used to predict protein fitness. Protein fitness may be a fitness score that identifies fitness of a protein for a particular function. The function may be how well a protein serves as part of a vaccine to certain virus, a catalyst, etc. Proteins that have high fitness, which is a fitness score above a predefined threshold, have numerous pharmaceuticals applications, vaccine formulations, etc. Further, identifying proteins with high fitness using embodiments discussed herein may reduce the time for identifying the protein from years to a matter of days or months, providing both cost and time savings.

Natural evolution provides a useful starting point for predicting protein fitness. The vast majority of possible sequences of amino acids have no biological function and would not fold properly to form a three-dimensional structure. In contrast, the small space of proteins that exist in nature generally do fold properly and perform some biological function. Unsupervised generative models have shown the ability to learn from evolution to predict protein fitness without access to labeled data. By learning the space of protein sequences that are naturally plausible, these models can assign scores based on model likelihoods or energies that often correlate with the true underlying fitness of proteins to perform their intended function.

While natural evolution provides a useful starting point, engineering proteins with novel or enhanced function beyond what exists in nature generally would require using assay-labeled protein sequences, where the fitness of a small number of proteins is measured in the laboratory. Assay-labeled measurements are expensive to obtain. Therefore, effective few-shot learning, where a model can learn from a small number of labeled examples, is a beneficial and pertinent hurdle to address. Specifically, strategies to adapt models that learn from evolution in the unsupervised setting via transfer learning to the downstream task of predicting fitness in the few-shot setting will be increasing important for protein fitness prediction.

For example, traditional approaches for unsupervised transfer learning in NLP pretrain a language model (LM) on unlabeled sequences, and reuse the hidden layers of the neural network in combination with a new randomly initialized output layer. Similar approaches also exist for protein fitness prediction, but throw away information. For example, the final linear language modeling head is able to predict the likelihood of possible candidate residues (amino acids), but this information is discarded when the language model is reinitialized with a regression head. The likelihoods of amino acid tokens under language model have been shown to be useful for making zero-shot protein fitness predictions, and to generate functional proteins, i.e. proteins having high fitness. Ideally, a language model should retain this likelihood information when finetuning to downstream fitness prediction tasks.

Thus, embodiments described herein use generative fitness finetuning (gf-tuning) as an approach to reuse the full probability distribution learned during unsupervised training to finetune to assay labeled data. Generative fitness tuning trains a generative model to use its likelihoods to classify the relative fitness of sequence pairs, allowing the model to directly reuse information learned from pretraining on the relative likelihoods of tokens. In this way, as information learnt during pretraining has been reused, a stronger fitness prediction performance may be achieved.

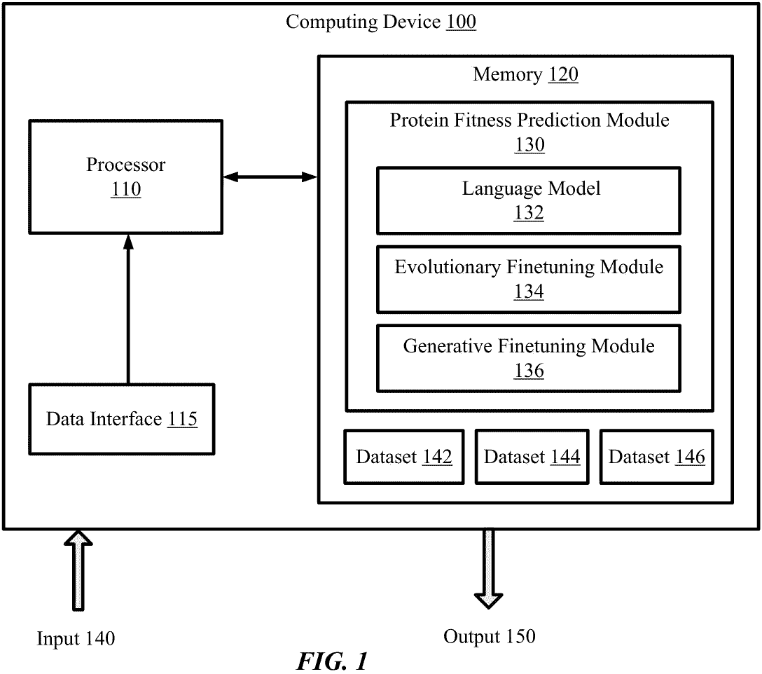

FIG. 1 is a simplified diagram of a computing device that implements the few-shot protein fitness prediction framework, according to some embodiments described herein. As shown in FIG. 1, computing device 100 includes a processor 110 coupled to memory 120. Operation of computing device 100 is controlled by processor 110. And although computing device 100 is shown with only one processor 110, it is understood that processor 110 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in computing device 100. Computing device 100 may be implemented as a stand-alone subsystem, as a board added to a computing device, and/or as a virtual machine.

Memory 120 may be used to store software executed by computing device 100 and/or one or more data structures used during operation of computing device 100. Memory 120 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Processor 110 and/or memory 120 may be arranged in any suitable physical arrangement. In some embodiments, processor 110 and/or memory 120 may be implemented on a same board, in a same package (e.g., system-in-package), on a same chip (e.g., system-on-chip), and/or the like. In some embodiments, processor 110 and/or memory 120 may include distributed, virtualized, and/or containerized computing resources. Consistent with such embodiments, processor 110 and/or memory 120 may be located in one or more data centers and/or cloud computing facilities.

In some examples, memory 120 may include non-transitory, tangible, machine readable media that includes executable code that when run by one or more processors (e.g., processor 110) may cause the one or more processors to perform the methods described in further detail herein. For example, as shown, memory 120 includes instructions for a few-shot protein fitness prediction framework. The few-shot protein fitness prediction framework includes a protein fitness prediction module 130 that may be used to implement and/or emulate the systems and models, and/or to implement any of the methods described further herein. In some examples, the protein fitness prediction module 130, may receive an input 140, e.g., protein sequences, which may be a sequence of amino acids, via a data interface 115. The data interface 115 may be any of a user interface that receives a user entered or uploaded sequence, or a communication interface that may receive or retrieve a previously stored sequence from memory 120 or another memory storage such as the database. The protein fitness prediction module 130 may generate an output 150, such as a classification of the protein sequence, a fitness prediction to the protein sequence, etc.

In some embodiments, the protein fitness prediction module 130 may include a language module 132, an evolutionary finetuning module 134 and a generative finetuning module 136. The language module 132 may a language model or the like that is pre-trained on some natural language dataset. The evolutionary finetuning module 134 and generative finetuning module 136 may finetune the pre-trained language module 132 to sequence regression and classification tasks by re-training the pre-trained language module 132 with a classification layer or regression head on top of the features of the final layer of the network. In one embodiment, the protein fitness prediction module 130 and its submodules 132-136 are implemented by hardware, software and/or a combination thereof.

The protein fitness prediction module 130 may train language model 132 using various datasets, such as datasets 142, 144, and 146. Dataset 142, referred to as dataset D_{f} may be a few-shot dataset of sequences x labeled with continuous fitness values y, D_{f} = {(x⁽¹⁾y⁽¹⁾) , _{.} , (x(^{|Df|}) , y(^{|Df|})}. The data in dataset D_{f} was acquired by applying mutations to a wildtype protein, e.g. protein that exists in nature, and obtaining fitness labels y for these mutants in laboratory.

Dataset 144 may be a large pretraining dataset of unlabeled protein sequences Dᵤ = {x⁽¹⁾,..,x(^{|Du|})}. Dataset 144 may be referred to as dataset Dᵤ.

Dataset 146 may be a smaller dataset of proteins that are evolutionarily related to the wildtype protein, Dₑ = {x⁽¹⁾,..., x^{(|De|)}}. Dataset 146 may be referred to as dataset Dₑ.

In some embodiments, sizes of datasets 142, 144, and 146 sizes may have a relationship where |Dᵤ| > |Dₑ| > |D_{f}|. The protein fitness prediction module 130 may train language model 132 to learn a predictive function

$$\hat{\text{y}} = F \left(x\right)$$

that results in a high correlation between ŷ and the ground truth fitness labels y. Once language model 132 is trained using protein fitness prediction module 130, the language model 132 may be evaluated using Spearman(ŷ, y), which determines a rank correlation equivalent to the Pearson correlation of the rank variables. This measures the ability of the language model 132 to accurately rank held out proteins by fitness.

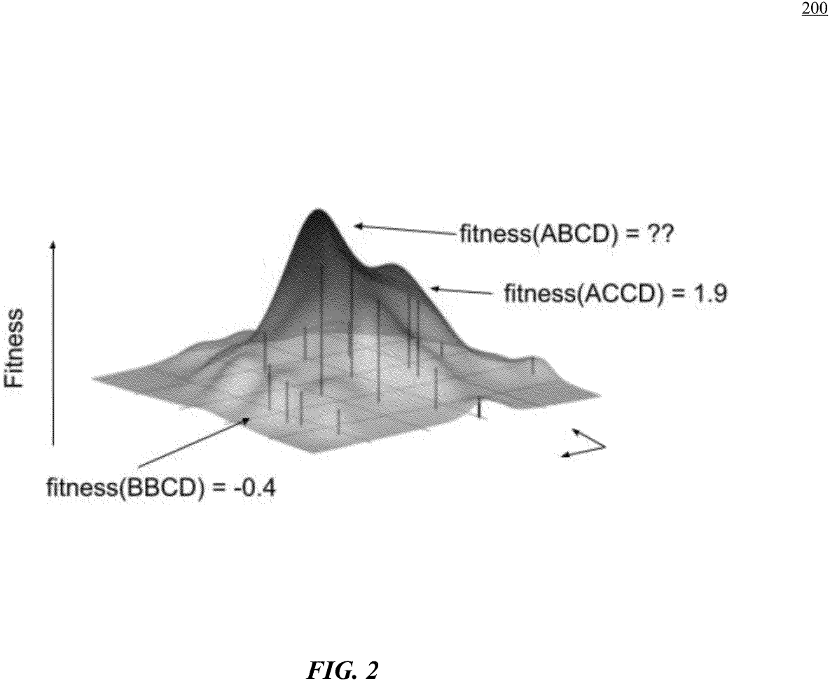

FIG. 2 is a simplified diagram 200 illustrating a protein fitness landscape, according to some embodiments. Protein fitness landscape describes how a set of mutations affect the function of a particular protein. Mutations may be a change in one or more of amino acids in a protein sequence. Suppose there is a simplified protein sequence BACD. FIG. 2 illustrates protein fitness for a simplified mutation protein sequence BBCD as -0.4 and protein fitness for a simplified mutation protein sequence ACCD as 1.9. Both BBCD and ACCD may be mutations of the simplified protein sequence BACD. The protein fitness prediction module 130 discussed herein may train language module 132 to identify protein fitness of mutations, such as protein sequence ABCD. As illustrated in FIG. 2, protein fitness may correspond to a fitness score. Further high and low fitness scores may be scores above and below predefined thresholds.

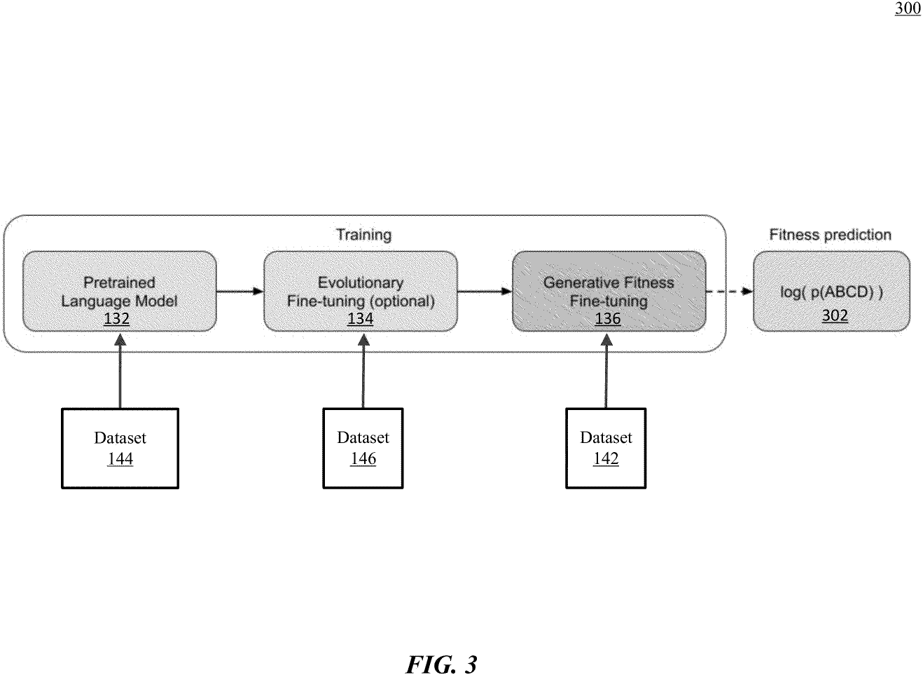

Going back to FIG. 1, in some embodiments, protein fitness prediction module 130 may finetune language model 132 using evolutionary finetuning module 134 and generative finetuning module 136. FIG. 3 is a simplified block diagram 300 of a protein fitness prediction module, according to some embodiments. The language model 132 may be optionally finetuned using with evolutionary finetuning using evolutionary finetuning module 134 and/or generative fitness finetuning using generative finetuning module 136. Once trained language module 132 may generate protein fitness prediction, which may be a fitness score 302 for various protein sequences and/or protein sequence mutations, such as protein ABCD shown in FIG. 2.

In some embodiments, evolutionary finetuning module 134 may use unsupervised fitness prediction. Before language model 132 is finetuned, language model 132 can perform protein fitness prediction without training on labeled data by learning to model the probability distribution over natural protein sequences. A probability distribution P_{θ} (x) can be pretrained to fit a large database of proteins, such as dataset 144, using the cost function L(Dᵤ) = -E_{x∼Dᵤ} [logP_{θ}(x)].

Once language model 132 is pre-trained, evolutionary finetuning module 134 may finetune language model 132 to evolutionary related sequences on dataset 146. Evolutionary finetuning module 134 may fit the pretrained unsupervised language model 132 to the cost function

$$L \left(( {D}_{e} ) = {x \sim {D}_{e}}_{} [ log {P}_{θ} ( x ) ]\right) .$$

Since protein sequences in dataset Dₑ (dataset 146) generally have a related protein function to the downstream task, finetuning to these protein sequences can improve the unsupervised fitness prediction ability of the pretrained generative model.

Language model 132 trained using evolutionary finetuning module 134 may learn the distribution of valid proteins for a particular protein family and may assign higher probabilities to valid proteins than invalid ones, thus predicting effect of mutations. Further, pre-trained language model 132 may give a strong initialization and useful inductive biases. When trained using evolutionary finetuning module 134, the language model 132 is finetuned to labeled data to leverage those inductive biases in the most effective way.

In some embodiments, evolutionary finetuning module 134 may finetune language model 132 with a regression head. For example, evolutionary finetuning module 134 may finetune a pre-trained language model 132 to sequence regression and classification tasks by retraining the neural network of the language model 132 with a classification or regression head on top of the features of the final layer of the neural network that makes up the language model 132. As discussed above, language model 132 may be pre-trained by maximizing the log-probability of natural protein sequences under the language model 132. For example, language model 132 may be pretrained to map a sequence x_{1:T} = {x₁, . ., xt, .., xT}, where T is the sequence length, to a set of corresponding hidden states h_{1:T}, where each hₜ ∈ ℝ^{d} and d is the hidden state dimensionality. During pretraining, a linear head W ∈ R^{|v|×d} is used to predict a distribution over the output vocabulary V for each time step, given by softmax(Whₜ). The output distribution can be trained to predict the next token xₜ₊₁ as in autoregressive language modeling if there is a left-to-right dependency in the predictive function, or recover masked xₜ tokens as in masked language modeling.

During finetuning, the sequence h_{1:T} is pooled down to a single hidden state vector hₚₒₒₗ ∈ ℝ^{d} using a pooling function. The pooling function uses the mean or max of each feature across the sequence, or simply uses the hidden state at a certain sequence position or special token. A regression head is then applied on top of this pooled feature representation of the sequence. The regression head can be a neural network, or simply a linear output layer. With a linear regression head, the prediction is given by taking the inner product of a learnable parameter vector w with the pooled sequence features via ŷ = w^{T}hₚₒₒₗ. The neural network of language model 132 is then finetuned to predict fitness values via the mean squared error loss function between y and ŷ. The training may occur until the loss function is minimized. The full neural network can be trained to predict protein fitness end to end, or the embedding of the neural network can be used as features for another model. Finetuning with a regression head throws away probabilistic information learned by the language model’s output layer during unsupervised pretraining about which sequences are more plausible, which has proven to be useful in unsupervised fitness prediction.

In some embodiments, evolutionary finetuning module 134 may finetune language model 132 with a linear regression-augmented density models. Linear regression-augmented density models reuse probabilistic information from the unsupervised pretraining by using the log-likelihood from the generative language model 132 as a feature for linear regression. In linear regression-augmented density model finetuning, evolutionary finetuning module 134 assumes that the sequences in dataset D_{f} (dataset 142) are all aligned to be the same length. Evolutionary finetuning module 134 uses linear ridge regression on the one hot amino-acid representation with an additional feature given by the density of a generative model 132 that has been trained on unsupervised data. Given density weight β, and embeddings for each position w_{1:T} where each wₜ ∈ ℝ^{|v|}, the predictions given by an augmented density model are given by:

$$\hat{\text{y}} \text{=} β log p \left(x\right) + {\sum }_{t = 1}^{T} {w}_{t}^{⊤} {e}_{{x}_{t}} ,$$

where eₓₜ gives the one-hot encoding of a residue at position t. The language model 132 is fit using ridge regression where the regularization parameter for β is set to be significantly lower than for w_{1:T}, forcing the linear model to rely more on the density to make its prediction.

In some implementations, a large scalar value is multiplied by log p(x) so that the value of β learned can be much smaller, reducing the effect of regularization on this feature. For baselines with augmented density models, log p(x) may be multiplied by 1000 to allow the ridge regression parameter to be shared for all weights, while having little to no effect on regularization of β since all fitness values in our experiments are much smaller than 1000 log p(x) for all models, allowing the learned β parameter to be used to predict fitness while having a very small value.

In some embodiments, evolutionary finetuning module 134 may finetune language model 132 using wildtype residual regression. For example, evolutionary finetuning module 134 may finetune masked language model 132 to fitness prediction using the sum of residuals between mutant log probabilities and wildtype log probabilities at mutation positions as predictions for mutational effect for regression. For example, evolutionary finetuning module 134 may finetune language model 132 using mutant marginal probabilities, where predictions are conditioned on the mutant sequence, such as:

$$\hat{\text{y}} = {\sum }_{t ∈ M} l o g {P}_{θ} \left(\left({x}_{t}^{m t}\right| {x}^{m t}\right) - l o g {P}_{θ} \left(\left({x}_{t}^{w t}\right| {x}^{m t}\right) .$$

where x^{m} is a mutant sequence and x^{w} is a wildtype sequence.

Evolutionary finetuning module 134 may the apply a mean squared error loss to

$$L \left({D}_{f}\right) = {\text{E}}_{\left(x , y\right) \sim {D}_{f}} \left[{\left({\hat{y}}^{\left(i\right)} - \left({y}^{\left(i\right)} - {y}^{w t}\right)\right)}^{2}\right] .$$

Masked language model 132 applied to mutational effect prediction assume additive effects of mutations, which would likely lead to non-optimal performance for epistatic proteins. Applying a mean squared error loss to residuals may also force the language model 132 to unlearn useful information learned during pretraining, as the unsupervised model will generally have a high mean squared error loss at initialization.

In some embodiments, generative finetuning module 136 may perform a generative fitness finetuning on the language model 132. The language model 132 may be a pre-trained language model 132 or language model 132 finetuned using evolutionary finetuning module 134. Generative finetuning module 136 may repurpose the probability distribution learned during unsupervised training as a pairwise classifier to classify the relative fitness of protein sequence pairs. Generative finetuning module 136 trains an auto-regressive language model 132 to model probability distribution P_{θ} (x), which computes the probability distribution as follows:

$${P}_{θ} \left({x}_{1 : T}\right) = {\prod }_{t = 1}^{T} {P}_{θ} \left(\left({x}_{t}\right| {x}_{< t}\right) ,$$

where T is the sequence length of sequence x_{1:T}. Using an auto-regressive language model 132 allows the language model 132 to be better suited for modelling the joint distribution when predicting multiple mutations as compared with a masked language model, which could be expected to be helpful for epistatic proteins since auto-regressive language models do not assume additive effects of mutations.

In some embodiments, language model 132 may be initialized as ProGen, an autoregressive language model that was pretrained on 280 million proteins. In settings with more evolutionarily related proteins, e.g., proteins that have high homology, ProGen may be adapted to evolutionarily related sequences with evolutionary finetuning. Generative finetuning module 136 may finetune the language model 132 initialized as ProGen to assay labeled data. Notably, generative finetuning module 136 may train any language model 132, including auto-regressive language model 132 to any parameterized probability distribution over protein sequences.

Generative finetuning module 136 may use a cost function P_{θ}(^{x(i)}) to classify whether the fitness of a randomly selected sequence xᵢ is higher than the fitness of a randomly selected sequence Xⱼ. Generative finetuning module 136 may use the probability density given by the language model 132 to make predictions, which classifies sequences with a higher probability as being higher fitness. The training cost function L(D_{f}) for generative fitness tuning may be as follows:

$$\left({x}^{\left(i\right)} , {y}^{\left(i\right)}\right) , \left({x}^{\left(j\right)} , {y}^{\left(j\right)}\right) ∼ {D}_{f}$$

$${P}_{θ} \left({y}^{( i )} > {y}^{( j )}\right) = \frac{{P}_{θ} {\left({x}^{\left(i\right)}\right)}^{α / {T}_{i}}}{{P}_{θ} {\left({x}^{\left(i\right)}\right)}^{α / {T}_{i}} + {P}_{θ} {\left({x}^{\left(j\right)}\right)}^{α / {T}_{j}}}$$

$${\overline{y}}_{i j} = \left\{\begin{matrix}1 , & i f {y}^{\left(i\right)} > {y}^{( j )} \\ 0.5 , & i f {y}^{\left(i\right)} = {y}^{( j )} \\ 0 , & o t h e r w i s e\end{matrix}\right)$$

$$L \left({D}_{f}\right) = \left[- {\overline{y}}_{i j} log {P}_{θ} \left({y}^{\left(i\right)} > {y}^{\left(j\right)}\right) - \left(1 - {\overline{y}}_{i j}\right) log \left(1 - {P}_{θ} \left({y}^{\left(i\right)} > {y}^{\left(j\right)}\right)\right)\right]$$

where a is a hyper-parameter. The method of scoring for pairwise comparisons may be based on the Bradley-Terry model. The generative finetuning module 136 may train generative model P(x) (model 132) to assign scores to x. The intuition is that the probability assigned to P_{θ}(y⁽ⁱ⁾ > y^{(j)}) will be equivalent to the likelihood of observing xᵢ before xᵢ if drawing samples from P_{θ}(x⁽ⁱ⁾) in the case where a = Tᵢ = Tⱼ.

Language model 132 trained using protein fitness prediction module 130 is suited for the downstream tasks and may be able to correctly classify most protein pairs without any supervised training. Accordingly, language model 132 may have an advantage at initialization over methods that use a randomly initialized regression head. Further, generatively finetuning language model 132 with this objective may improve ability of language model 132 to perform classification, thus allowing language model 132 to assign scores that can be better used to rank proteins by fitness.

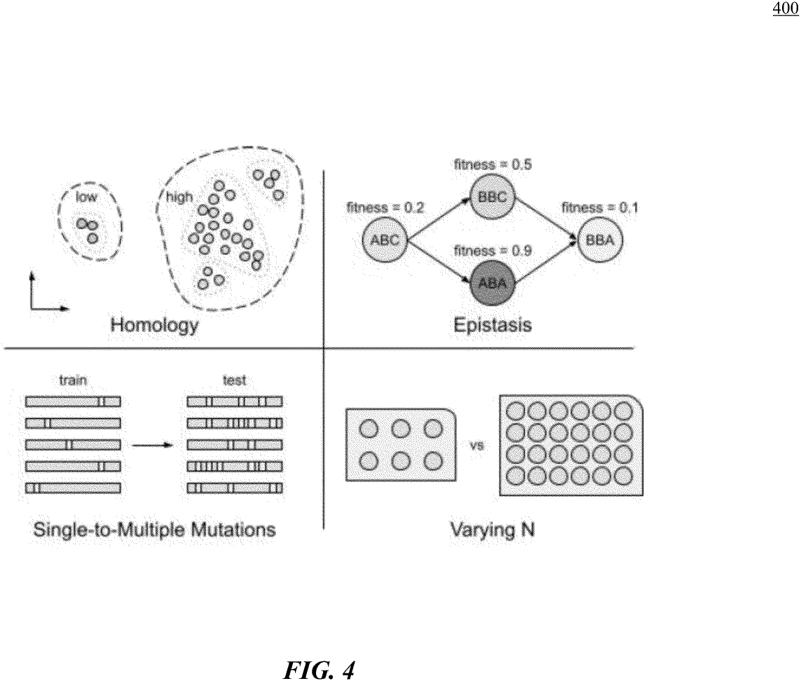

FIG. 4 is a simplified diagram 400 illustrating an example of the protein fitness prediction tasks, according to some embodiments. As shown in FIG. 4, protein fitness prediction tasks may be subdivided based on different problem settings. The number of homologous sequences, level of epistasis, extrapolation to higher edit distances, and amount of labeled data in few-shot scenarios are factors to consider for fitness modeling. Different few-shot protein fitness prediction problems can have very different characteristics, and language model 132 may be trained to capture the range of practical scenarios with choice of tasks and data sets.

One task may determine protein fitness in the high homology and low homology protein domain. High homology protein domains have more evolutionarily related sequences that evolved to perform a similar function, and can potentially act as more useful unlabeled sequences for unsupervised training. Unsupervised conventional models are therefore expected to perform better in high homology domains, and few-shot models that can better leverage an unsupervised initialization are at an advantage. Low homology protein domains do not have as many evolutionarily related proteins that perform a similar function, but can still leverage general pretraining across protein databases of many families.

A homology of a protein may be approximated using a number sequences in the multiple sequence alignment (MSA) of evolutionarily related proteins. Since many proteins in databases can be very small edit distances away from each other, sequence diversity is an important consideration for homology determination. Therefore, the total number of clusters for sequence clustering with a 50% sequence identity threshold provides approximately 80% coverage in sequence alignment using mmseqs2 as another metric.

Trained language model 132 can process tasks with varying degrees of epistatic. In epistatic protein domains, a mutation at one position of the protein can greatly influence the mutational effect of a mutation at another position. An additive model can approximate protein fitness landscapes. The additive model may predict the mutational effect of mutation A and mutation B together to be the sum of the mutational effect of mutation A and mutation B to be non-epistatic. Protein fitness landscapes cannot be modeled well with an additive model to be epistatic. Epistatic fitness prediction problems are difficult because they require predicting nonlinear interactions between mutations to perform well. To score the level of epistasis definition, the test set in Spearman may be used to predict correlation of an additive model that uses the effects of all single mutations to predict multiple mutations as a metric to approximate the inverse of epistatic of a protein fitness prediction task.

Language model 132 may be evaluated using tasks on two different kinds of train/test splits: First, training and test set are randomly distributed. Second training occurs on single mutants only, and testing occurs on multiple mutants only. Training on single mutants and evaluating on multiple mutants requires the model to generalize beyond its training set. Single mutation synthesis and assay data can also be easier to perform and acquire in the laboratory, so a model that can generalize from single to multiple mutations is of practical use.

Language model 132 may evaluate tasks in the few-shot scenario because obtaining labeled data for protein fitness is expensive and language model 132 that performs well in few-shot scenarios are useful in practice. Test set performance may be considered for several different training set sizes for each model, such as n = 48, 96, and 240. Leveraging unsupervised pretraining is more important for smaller training set sizes, as there is less information that can be learned during supervised finetuning.

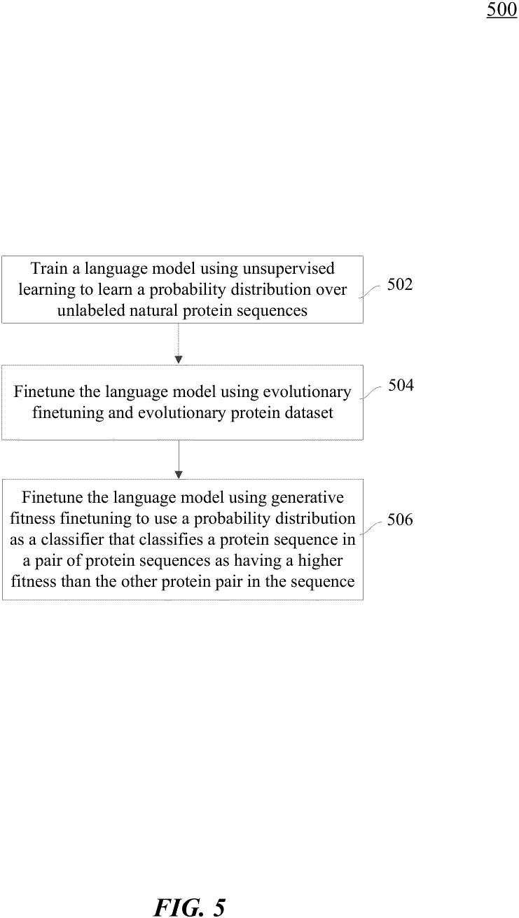

FIG. 5 is a simplified diagram of a method 500 for training a pre-trained language model using a protein fitness prediction module, according to some embodiments. One or more of the processes 502-508 of method 500 may be implemented, at least in part, in the form of executable code stored on non-transitory, tangible, machine-readable media that when run by one or more processors may cause the one or more processors to perform one or more of the processes 502-508.

At process 502, a language model is trained using unsupervised learning. For example, language model 132 is a generative language model that is trained on training dataset 144 of unlabeled protein sequences. Language model 132 is trained to learn a probability distribution P_{θ} (x) over natural protein sequences. Language model 132 may be trained using loss function L(Du) = -E_{x∼Dᵤ}[logP_{θ}(x)].

At process 504, a language model is finetuned using evolutionary finetuning. For example, language model 132 may be evolutionary finetuned using evolutionary protein dataset 146. In some embodiments, process 504 may be optional.

At process 506, a language model is finetuned using generative fitness finetuning. For example, language model 132 may be finetuned by training the probability distribution learned during process 502 as a pairwise classifier. During generative finetuning, language model 132 receives pairs of protein sequences as input. Language model 132 is trained to classify which of the protein sequences in the pairs has a higher fitness. The classification of the protein sequences is then used to determine a value for the loss function, such as the loss function in Eq. 7. Process 506 may repeat until the loss function is minimized.

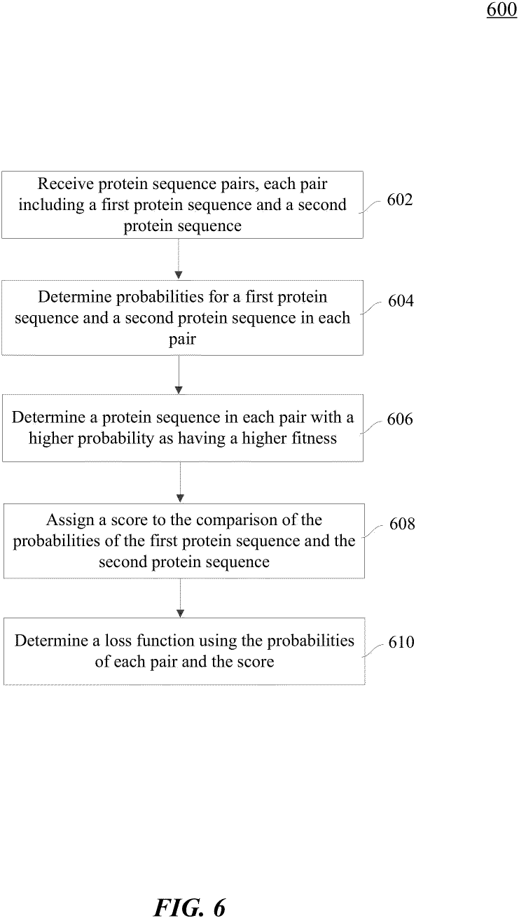

FIG. 6 is a simplified diagram of a method 600 for finetuning the language model using generative fitness finetuning, according to some embodiments. One or more of the processes 602-610 of method 600 may be implemented, at least in part, in the form of executable code stored on non-transitory, tangible, machine-readable media that when run by one or more processors may cause the one or more processors to perform one or more of the processes 602-610. Further, process 602-610 may be performed iteratively until language model 132 is finetuned.

At process 602, a language model is trained using protein sequence pairs. The protein sequences in the protein sequence pairs may be randomly selected from dataset 142. The language model 132 receives protein sequence pairs, where each pair includes a first protein sequence and a second protein sequence;

At process 604, probabilities for the protein sequences in the protein sequence pair are determined. For example, language model 132 is trained to determined probability of the first protein sequence and probability of the second protein sequence using the probability distribution. Process 604 may repeat for each protein sequence pair.

At process 606, a protein sequence with a higher probability is determined as having a higher fitness. For example, language model 132 is trained to determine whether the first protein sequence or the second protein sequence has a higher probability, and hence higher fitness.

At process 608, a score is assigned to the comparison of the probabilities of the first protein sequence and the second protein sequence. The score may be determined using Eq. (6), discussed above. The score is determined for each pair received in process 602.

At process 610, a value for the loss function is determined using the probabilities of the first protein sequence, the second protein sequence and the score. The value for the loss function may be determined using the loss function in Eq. (7).

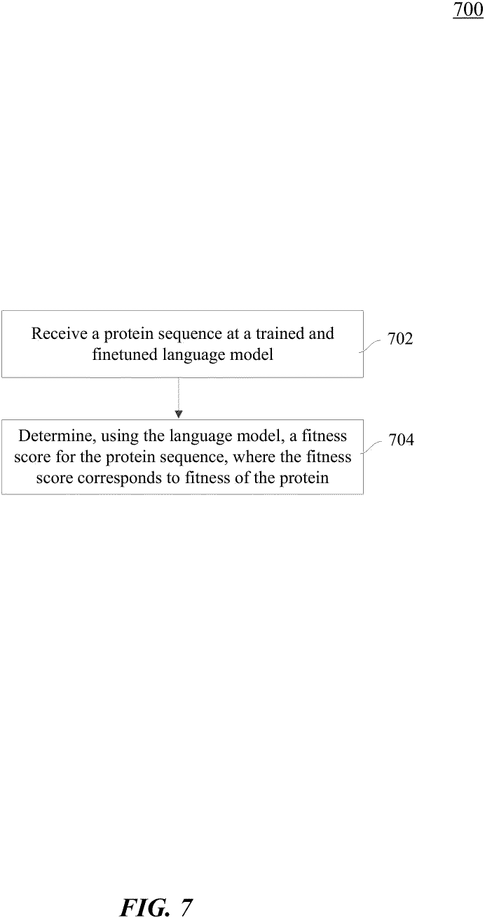

FIG. 7 is a simplified diagram of a method 700 for predicting protein fitness of a protein sequence, according to some embodiments. One or more of the processes 702-704 of method 700 may be implemented, at least in part, in the form of executable code stored on non-transitory, tangible, machine-readable media that when run by one or more processors may cause the one or more processors to perform one or more of the processes 702-704.

At process 702, a protein sequence is received. For example, language model 132 that is trained and finetuned as discussed in FIGS. 5-6, receives a protein sequence as input 140.

At process 704, a fitness score is generated. For example, language model 132 may generate a fitness score 302 for the protein sequence received in process 602. The fitness score 302 may be an output 150. As discussed above a fitness score 302 is indicative of a fitness of the protein sequence. Fitness is a measure of a protein to function or perform as desired. For example, high-fitness may satisfy all of the criteria for the protein sequence to function as desired, or at least to perform well in the assay used for screening. Fitness may, for example, include the ability of the protein sequence to recognize one substrate but not another, to be expressed at high levels in a particular host organism, to not aggregate, to have a long life-time, and etc.

Some examples of computing devices, such as computing device 100 may include non-transitory, tangible, machine readable media that include executable code that when run by one or more processors (e.g., processor 110) may cause the one or more processors to perform the processes of methods discussed above. Some common forms of machine-readable media that may include the processes of method are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

This description and the accompanying drawings that illustrate inventive aspects, embodiments, implementations, or applications should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure the embodiments of this disclosure. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

What is claimed is:

1. A method for training a language model to predict a protein fitness score for a protein sequence, the method comprising:
training the language model on a training dataset of unlabeled protein sequences to learn a probability distribution over protein sequences in the training dataset; and
finetuning the language model by training the probability distribution learned during training as a pairwise classifier that classifies a relative fitness of protein sequence pairs until a loss function is minimized, wherein the finetuning further comprises:
receiving a protein sequence pair of the protein sequence pairs, the protein sequence pair including a first protein sequence and a second protein sequence;
determining, using the probability distribution, a first probability of the first protein sequence and a second probability of the second protein sequence;
selecting a protein sequence from the first protein sequence and the second protein sequence that corresponds to a higher probability between the first probability and the second probability as the protein sequence with a higher fitness; and
determining a value of the loss function based on the selecting the protein sequence.

2. The method of claim 1, wherein the language model is a generative language model.

3. The method of claim 1, wherein the value of the loss function includes the first probability, the second probability, and a score based on a first fitness value of the first protein sequence and a second fitness value of the second protein sequence.

4. The method of claim 3, wherein determining the score further comprising:
determining the first fitness value using the first probability of the first protein sequence;
determining the second fitness value using the second probability of the second protein sequence; and
determining the score based on first fitness value and the second fitness value.

5. The method of claim 1, wherein the training dataset further includes fitness labels.

6. The method of claim 1, further comprising:
generating a fitness score for a new protein sequence using the trained and finetuned language model.

7. The method of claim 1, further comprising:
selecting the protein sequence pairs from a few-shot protein dataset, wherein sequences in the few-shot protein dataset are variants of protein sequences that exist in nature.

8. A system for training a language model to predict a protein fitness score for a protein sequence, the system comprising:
a memory configured to store the language model and a protein fitness prediction module; and
a processor coupled to the memory and configured to cause the protein fitness prediction module to perform operations, the operations comprising:
training the language model on a training dataset of unlabeled protein sequences to learn a probability distribution over protein sequences in the training dataset; and
finetuning the language model by training the probability distribution learned during training as a pairwise classifier that classifies a relative fitness of protein sequence pairs until a loss function is minimized, wherein the finetuning further comprises:
receiving a protein sequence pair of the protein sequence pairs, the protein sequence pair including a first protein sequence and a second protein sequence;
determining, using the probability distribution, a first probability of the first protein sequence and a second probability of the second protein sequence;
selecting a protein sequence from the first protein sequence and the second protein sequence that corresponds to a higher probability between the first probability and the second probability as the protein sequence with a higher fitness; and
determining a value of the loss function based on the selecting the protein sequence.

9. The system of claim 8, wherein the language model is a generative language model.

10. The system of claim 8, wherein the value of the loss function includes the first probability, the second probability, and a score based on a first fitness value of the first protein sequence and a second fitness value of the second protein sequence.

11. The system of claim 10, wherein the operations for determining the score further comprise:
determining the first fitness value using the first probability of the first protein sequence;
determining the second fitness value using the second probability of the second protein sequence; and
determining the score based on first fitness value and the second fitness value.

12. The system of claim 8, wherein the operations further comprise:
selecting the protein sequence pairs from a few-shot protein dataset, wherein sequences in the few-shot protein dataset are variants of protein sequences that exist in nature.

13. The system of claim 8, further comprising:
selecting the protein sequence pairs from a few-shot protein dataset, wherein sequences in the few-shot protein dataset are variants of protein sequences that exist in nature.

14. The system of claim 8, further comprising:
generating a fitness score for a new protein sequence using the trained and finetuned language model.

15. A non-transitory computer readable medium having instructions stored thereon, that when executed by a processor causes the processor to perform operations, the operations comprising:
training a language model on a training dataset of unlabeled protein sequences to learn a probability distribution over protein sequences in a training dataset; and
finetuning the language model by training the probability distribution learned during training as a pairwise classifier that classifies a relative fitness of protein sequence pairs, wherein the finetuning further comprises:
receiving a protein sequence pair of the protein sequence pairs, the protein sequence pair including a first protein sequence and a second protein sequence;
determining, using the probability distribution, a first probability of the first protein sequence and a second probability of the second protein sequence; and
selecting a protein sequence from the first protein sequence and the second protein sequence that corresponds to a higher probability between the first probability and the second probability as the protein sequence with a higher fitness.

16. The non-transitory computer readable medium of claim 15, further comprising:
finetuning, using a loss function, the language model, wherein the loss function includes the first probability, the second probability, and a score based on a first fitness value of the first protein sequence and a second fitness value of the second protein sequence.

17. The non-transitory computer readable medium of claim 16, wherein determining the score further comprises:
determining the first fitness value using the first probability of the first protein sequence;
determining the second fitness value using the second probability of the second protein sequence; and
determining the score based on first fitness value and the second fitness value.

18. The non-transitory computer readable medium of claim 15, further comprising:
generating a fitness score for a new protein sequence using the trained and finetuned language model.

19. The non-transitory computer readable medium of claim 15, wherein the training dataset further includes fitness labels.

20. The non-transitory computer readable medium of claim 15, further comprising:
selecting the protein sequence pairs from a few-shot protein dataset, wherein sequences in the few-shot protein dataset are variants of protein sequences that exist in nature.
